(19) United States
(12) Patent Application Publication
(10) Pub. No.: US 2015/0111264 A1
(43) Pub. Date: Apr. 23, 2015

(54) Method for Producing Lipid

(71) Applicant: Kao Corporation, Chuo-ku, Tokyo (JP)

(72) Inventors: Tatsuro Ozaki, Wakayama-shi (JP); Yasushi Takimura, Wakayama-shi (JP)

(73) Assignee: Kao Corporation, Chuo-ku, Tokyo (JP)

(21) Appl. No.: 14/396,990

(22) PCT Filed: Apr. 26, 2013
(86) PCT No.: PCT/JP2013/062441

§ 371 Date: Oct. 24, 2014

(30) Foreign Application Priority Data
May 8, 2012 (JP) 2012-106880
Dec. 14, 2012 (JP) 2012-273950

(51) Int. Cl. C12P 7/64; C07C 67/08

(52) U.S. Cl. CPC C12P 7/6409; C07C 67/08
USPC 435/134

(57) ABSTRACT

Provided is a method for producing a lipid, by which lipid including a high ratio of medium-chain fatty acids can be produced efficiently.

Disclosed is a method for producing a lipid, the method comprising the following steps (1) and (2): (1) a step of culturing algae of the class Dinophyceae in a medium containing glycerin to obtain a culture; and (2) a step of collecting a lipid from a culture thus obtained.

### FIELD OF THE INVENTION

The present invention relates to a method for producing a lipid using algae of the class Dinophyceae.

### BACKGROUND OF THE INVENTION

Medium-chain fatty acids, which are represented by lauric acid, are principal fatty acids that are contained in large amounts in coconut oil or palm kernel oil, and are used as the raw materials of various surfactants or foods, for example. Among the medium-chain fatty acids, the supply source of lauric acid is limited to coconut and palm kernels, which are grown in limited areas in the world. Furthermore, when arable lands are used to obtain the raw material of medium-chain fatty acids, there is a risk that this use may compete with use for biodiesel or food products in the future. There also comes along the problem of destruction of tropical rain forest. Therefore, there has been a demand for the development of a technology for supplying medium-chain fatty acids, which does not depend on coconut or palm kernel.

On the other hand, it is known that Crypthecodinium cohnii, a dinoflagellate, contains a high ratio of lauric acid (see Non-Patent Literature 1).

Furthermore, the present applicant found that an oil or fat having a content percentage of lauric acid of 3% by mass or more in the fatty acid composition can be produced by culturing algae of the genus Symbiodinium in a medium, and filed a patent application (see Patent Literature 1).

However, since it is predicted that the demand of medium-chain fatty acids for biodiesel or food materials will be increased in the future, it is desired to further develop a method for producing a lipid including a high ratio of medium-chain fatty acids.

### CITATION LIST

### Patent Literature

Patent Literature 1: WO 2011/108755 A

### Non Patent Literature

Non-Patent Literature 1: Phytochemistry, (1988) 27, 1679-1683

### SUMMARY OF THE INVENTION

An aspect of the present invention relates to a method for producing a lipid, the method comprising the following steps (1) and (2) (hereinafter, may be referred to as “production method of the present invention”):

(1) a step of culturing algae of the class Dinophyceae in a medium containing glycerin, to obtain a culture (hereinafter, may be referred to as “step 1”); and

(2) a step of collecting a lipid from the culture thus obtained (hereinafter, may be referred to as “step 2”).

Another aspect of the present invention relates to a method for producing medium-chain fatty acid esters, the method comprising a step of subjecting the lipid obtained by the production method of the present invention described above, to a transesterification reaction using an alcohol (hereinafter, may be referred to as “ester production method of the present invention”).

Another aspect of the present invention relates to a method for producing medium-chain fatty acids, the method comprising a step of hydrolyzing the lipid obtained by the production method of the present invention (hereinafter, may be referred to as fatty acid production method of the present invention”).

Still another aspect of the present invention relates to a method for enhancing the productivity of medium-chain fatty acids in the algae, the method comprising culturing algae of the class Dinophyceae in a medium containing glycerin.

Still another aspect of the present invention relates to a method for producing an alga having increased productivity of medium-chain fatty acids, the method comprising culturing algae of the class Dinophyceae in a medium containing glycerin.

### DETAILED DESCRIPTION OF THE INVENTION

The present invention relates to a provision of a method for producing a lipid, in which lipid, preferably an oil or fat, including a high ratio of medium-chain fatty acids can be produced efficiently.

The inventors of the present invention conducted further investigations on the production of lipid using algae of the class Dinophyceae, and they found that when the algae are cultured using glycerin among the various carbon sources that are usually added to media as nutrition sources for proliferating cells, unexpectedly, the amount of fatty acids is markedly increased without affecting cell proliferation, the content percentage of medium-chain fatty acids among all the constituent fatty acids in the lipid thus obtained (hereinafter, also referred to as “productivity of medium-chain fatty acids”) is significantly increased, and a lipid including a high ratio of medium-chain fatty acids can be efficiently produced.

According to the production method of the present invention, a lipid including a high ratio of medium-chain fatty acids can be produced efficiently. Furthermore, according to the ester production method of the present invention, an ester form of medium-chain fatty acid can be produced efficiently. In addition, according to the method for producing medium-chain fatty acids of the present invention, medium-chain fatty acids can be produced efficiently.

<Method for Producing Lipid>

(Lipid)

In the production method of the present invention, examples of lipid include a simple lipid that includes a fatty acid and an ester of a fatty acid and a variety of alcohol (for example, an oil or fat, and a wax); a complex lipid composed of, for example, a fatty acid, an alcohol, a phosphoric acid, a sugar and the like (for example, phospholipid and glycolipid); and a derivative lipid such as a hydrolysate of the two categories of lipids mentioned above, which is insoluble in water (for example, a fatty acid, a higher alcohol and a sterol), or terpenes and an oil-soluble vitamin. Among these, the lipid is preferably a simple lipid or a complex lipid, more preferably a simple lipid, even more preferably an oil or fat, from the viewpoint of increasing the productivity of medium-chain fatty acids.

(Oil or Fat)

In the production method of the present invention, the term “oil or fat” means an ester of fatty acid and glycerin, and specifically refers to a neutral lipid such as monoglyceride, diglyceride and triglyceride. Furthermore, a medium-chain fatty acid refers to a monovalent carboxylic acid having a hydrocarbon group having 10 to 14 carbon atoms, and specific examples thereof include capric acid, lauric acid and myristic acid. Among these, myristic acid and lauric acid are preferred, and lauric acid is more preferred. Furthermore, a fatty acid ester refers to an ester of a fatty acid and a lower or higher alcohol, other than the triglyceride, and a medium-chain fatty acid ester refers to an ester of the medium-chain fatty acid and a lower or higher alcohol.

(Step 1)

The production method of the present invention includes a step of culturing algae of the class Dinophyceae in a medium containing glycerin.

(Algae of Class Dinophyceae)

The algae of the class Dinophyceae used in the present invention taxonomically refer to a group of unicellular algae that perform photosynthesis and have flagella, the algae having sulcus and cingulum on the surface of the cell and having a characteristic form through plural symbioses in addition to the second symbiosis of red algae during their evolution. Specific examples of the algae of the class Dinophyceae include algae of the order Noctilucales, the order Prorocentrales, the order Dinophysales, the order Gymnodiniales, the order Peridiniales, the order Gonyaulacales, the order Blastodiniales, the order Phytodiniales and the like. Among these, from the viewpoint of increasing the productivity of medium-chain fatty acids, algae of the order Gymnodiniales are preferred.

Examples of the algae of the order Gymnodiniales include algae of the genera Amphidinium, Cochlodinium, Gymnodinium, Gyrodinium, Erythropsodinium, Hemidinium, Katodinium, Nematodinium, Oxyrrhis, Polykrikos, Torodinium, Symbiodinium, Warnowia, Woloszynskia, Zooxanthella and the like. Among these, from the viewpoint of increasing the productivity of medium-chain fatty acids, algae of the genus Symbiodinium are preferred.

Examples of the algae that belong to the genus Symbiodinium include Symbiodinium microadriaticum, Symbiodinium goreaui, Symbiodinium linucheae, Symbiodinium bermudense, Symbiodinium meandrinae, Symbiodinium californium, Symbiodinium kawagutii, Symbiodinium corculorum, Symbiodinium consortia, Symbiodinium muscatinei, Symbiodinium freudenthal, Symbiodinium pulchrorum, Symbiodinium pilosum and the like. Among these, from the viewpoint of increasing the productivity of medium-chain fatty acids-containing lipid, Symbiodinium microadriaticum is preferred, and Symbiodinium microadriaticum strain LB2281 and Symbiodinium sp. strain NIES-2638 are more preferred.

Meanwhile, the algae of the class Dinophyceae are available from public institutions such as the Culture Collection of Algae at University of Texas at Austin (UTEX), National Institute for Environmental Studies (NIES), National Center for Marine Algae and Microbiota (NCMA; formerly, CCMP) and Culture Collection of Algae and Protozoa (CCAP).

(Medium)

For the medium that is used to culture algae in Step 1, those conventionally known media can be used, medium based on natural seawater or artificial seawater may be used, and commercially available culture media may also be used. Preferred examples of the medium include Daigo IMK medium, f/2 medium, ESM medium, L1 medium, MNK medium and the like. Among these, from the viewpoint of increasing the productivity of medium-chain fatty acids and from the viewpoint of the concentration of nutrient components, f/2 medium, ESM medium, and Daigo IMK medium are preferred, ESM medium and Daigo IMK medium are more preferred, and Daigo IMK medium is even more preferred.

The medium used in Step 1 contains glycerin from the viewpoint of increasing the productivity of medium-chain fatty acids. Here, glycerin may include polyglycerin, which is a product of a polycondensation of glycerin. From the viewpoint of increasing the productivity of medium-chain fatty acids, the content of glycerin in the medium is preferably 0.01% by mass or more, more preferably 0.02% by mass or more, even more preferably 0.05% by mass or more, even more preferably 0.07% by mass or more, even more preferably 0.08% by mass or more, even more preferably 0.09% by mass or more, even more preferably 0.1% by mass or more. Also, from the same point of view, the content is preferably 10% by mass or less, more preferably 5% by mass or less, even more preferably 4% by mass or less, even more preferably 3% by mass or less, even more preferably 2% by mass or less, even more preferably 1% by mass or less, even more preferably 0.5% by mass or less. Furthermore, the content of glycerin in the medium is, from the viewpoint of increasing the productivity of medium-chain fatty acids, from 0.01% to 10% by mass, preferably from 0.02% to 5% by mass, more preferably from 0.05% to 4% by mass, even more preferably from 0.07% to 3% by mass, even more preferably from 0.08% to 2% by mass, even more preferably from 0.09% to 2.0% by mass, even more preferably from 0.1% to 2.0% by mass, even more preferably from 0.1% to 1% by mass, even more preferably from 0.1% to 0.5% by mass.

In the medium used in Step 1, for example, a nitrogen source, a phosphorus source, a metal salt, a vitamin, a carbon source other than glycerin or a trace metal can be added appropriately from the viewpoints of promoting the growth of algae and increasing the productivity for medium-chain fatty acids.

Examples of the nitrogen source include NaNO₃, KNO₃, Ca(NO₃)₂, NH₄NO₃, (NH₄)₂SO₄ and the like. Examples of the phosphorus source include K₂HPO₄, KH₂PO₄, Na₂HPO₄, NaH₂PO₄, sodium glycerophosphate and the like. Examples of the metal salt include NaCl, KCl, CaCl₂, MgCl₂, Na₂SO₄, K₂SO₄, MgSO₄, Na₂CO₃, NaHCO₃, Na₂SiO₃, H₃BO₃, MnCl₂, MnSO₄, FeCl₃, FeSO₄, CoCl₂, ZnSO₄, CuSO₄, Na₂MoO₄ and the like. Examples of the vitamin include biotin, vitamin B12, thiamine-HCl, nicotinic acid, inositol, folic acid, thymine and the like.

The content of the nitrogen source in the medium is, in terms of nitrogen atom equivalent, preferably 2 mg/L or more, more preferably 5 mg/L or more, even more preferably 10 mg/L or more, even more preferably 15 mg/L or more, even more preferably 20 mg/L or more, even more preferably 50 mg/L or more, even more preferably 100 mg/L or more, from the viewpoint of increasing the productivity of lipid and medium-chain fatty acids; and from the same point of view, the content of the nitrogen source is, in terms of nitrogen atom equivalent, preferably 700 mg/L or less, more preferably 600 mg/L or less, even more preferably 500 mg/L or less, even more preferably 400 mg/L or less, even more preferably 300 mg/L or less, even more preferably 250 mg/L or less. Furthermore, from the viewpoint of increasing the productivity of lipid and medium-chain fatty acids, the content of the nitrogen source in the medium is, in terms of nitrogen atom equivalent, from 2 mg/L to 700 mg/L, preferably from 5 mg/L to 600 mg/L, more preferably from 10 mg/L to 500 mg/L, even more preferably from 15 mg/L to 400 mg/L, even more preferably from 20 mg/L to 300 mg/L, even more preferably from 50 mg/L to 250 mg/L, even more preferably from 100 mg/L to 250 mg/L.

Furthermore, from the viewpoint of increasing the productivity of lipid and medium-chain fatty acids, the content of the phosphorus source in the medium is, in terms of phosphorus atom equivalent, preferably 0.5 mg/L or more, more preferably 1 mg/L or more, even more preferably 2 mg/L or more, even more preferably 4 mg/L or more, even more preferably 8 mg/L or more; and from the same point of view, the content of the phosphorus source is, in terms of phosphorus atom equivalent, 100 mg/L or less, more preferably 50 mg/L or less, even more preferably 25 mg/L or less, even more preferably 20 mg/L or less. Furthermore, from the viewpoint of increasing the productivity of lipid and medium-chain fatty acids, the content of the phosphorus source in the medium is, in terms of phosphorus atom equivalent, from 0.5 mg/L to 100 mg/L, more preferably from 1 mg/L to 50 mg/L, even more preferably from 2 mg/L to 25 mg/L, even more preferably from 4 mg/L to 20 mg/L, even more preferably from 8 mg/L to 20 mg/L.

Examples of the carbon source other than glycerin include carbon dioxide, acetic acid, sodium acetate, glucose, sucrose, fructose, starch, fatty acid and the like. When the carbon source other than glycerin in the medium is glucose, the content of glucose in the medium is preferably 5% by mass or less, more preferably 2% by mass or less, even more preferably 1% by mass or less, even more preferably 0.5% by mass or less, even more preferably 0.1% by mass or less, from the viewpoint of enhancing the productivity of medium-chain fatty acids.

The pH of the medium used in Step 1 is preferably appropriately selected depending on the kind of algae used, and from the viewpoints of suppressing the propagation of miscellaneous microorganisms, promoting the growth of algae and increasing the productivity of medium-chain fatty acids, the pH of the medium is preferably in the range of from 5 to 10, more preferably in the range of from 6 to 9, and even more preferably in the range of from 7 to 8. Furthermore, the pH of the medium at the time of culture is, from the same point of view, preferably from 6 to 9, more preferably from 7 to 8. The pH can be appropriately adjusted to a desired range by adding an acid or a base to the medium. Meanwhile, regarding the medium used, it is preferable to use the medium after sterilizing the medium by, for example, autoclaving or filtration through a filter, from the viewpoints of suppressing the propagation of miscellaneous microorganisms, promoting the growth of algae and increasing the productivity of medium-chain fatty acids.

(Culture Conditions)

The amount of algae to be inoculated into the medium is not particularly limited; and, from the viewpoint of growth, the amount of algae is preferably from 1% to 10% (vol/vol), more preferably from 1% to 5% (vol/vol), per medium.

The culture temperature for Step 1 is not particularly limited as long as the temperature is in the range that does not adversely affect the proliferation of the algae used; and, the culture temperature is usually in the range of from 5° C. to 40° C. From the viewpoints of promoting the growth of algae, increasing the productivity of medium-chain fatty acids and reducing the production cost, the culture temperature is more preferably from 10° C. to 30° C., even more preferably from 15° C. to 25° C.

In regard to the production method of the present invention, from the viewpoints of promoting the growth of algae and increasing the productivity of medium-chain fatty acids, it is preferable to carry out the culture of the algae under light irradiation. Regarding light irradiation, any conditions that enable photosynthesis may be used, and the light may be artificial light or sunlight. The illuminance at the time of light irradiation is preferably in the range of from 100 Lux to 50,000 Lux, more preferably in the range of from 300 Lux to 10,000 Lux, even more preferably in the range of from 1,000 Lux to 6,000 Lux, from the viewpoints of promoting the growth of algae and increasing the productivity of medium-chain fatty acids. Furthermore, the interval of light irradiation is not particularly limited; and, from the viewpoints of promoting the growth of algae and increasing the productivity of medium-chain fatty acids, it is preferable to carry out the light irradiation with the light-dark cycle, and the cycle is, from the same point of view as described above, preferably from 8 hours to 24 hours, more preferably from 10 hours to 18 hours, even more preferably 12 hours.

Regarding the culture for Step 1, the culture time is not particularly limited as long as the culture is carried out such that the algal bodies accumulating a lipid at a high concentration would proliferate at a high concentration, and for example, the culture may be carried out for a long time of about 150 days. From the viewpoints of promoting the growth of algae, increasing the productivity of medium-chain fatty acids and reducing the production cost, the culture period after the addition of glycerin is preferably 3 days or longer, more preferably 7 days or longer, and preferably 90 days or shorter, more preferably 56 days or shorter, even more preferably 49 days or shorter, even more preferably 35 days or shorter, even more preferably 30 days or shorter. For example, the culture period may be from 3 to 90 days, preferably from 3 to 30 days, more preferably from 7 to 30 days. Meanwhile, the culture may be carried out by any of aerated and agitated culture, shaking culture, or static culture; and, from the viewpoint of increasing air permeability, shaking culture is preferred.

(Culture)

The culture used in Step 1 means the algal bodies and the medium obtained after culturing algae. From the viewpoints of increasing the productivity of medium-chain fatty acids and reducing the production cost, the culture is preferably the algal bodies obtained after culturing algae.

(Step 2)

The production method of the present invention includes a step of collecting a lipid from the culture obtained by Step 1 described above.

The method for collecting a lipid from a culture is not particularly limited. For example, after the completion of culture, a lipid can be collected by separating the algal bodies from the medium, crushing the algal bodies thus obtained and then performing solvent extraction using an organic solvent.

Examples of the method for separating the algal bodies from the medium include filtration and centrifugation; and, from the viewpoint of reducing the production cost, filtration is preferred. Examples of the method for crushing the algal bodies include compression, ultrasonic crushing, enzymatic treatment and chemical treatment; and, from the viewpoint of reducing the production cost, compression is preferred.

Examples of the organic solvent used for the solvent extraction include chloroform, hexane, butanol, methanol and ethyl acetate. Among these, from the viewpoints of increasing the extraction efficiency of lipid and reducing the production cost, one kind or two or more kinds selected from the group consisting of chloroform, methanol, ethyl acetate and hexane are preferred, one kind or two or more kinds selected from the group consisting of ethyl acetate and hexane are more preferred, and hexane is even more preferred.

The weight ratio of the culture and the organic solvent (culture/organic solvent) at the time of extraction is preferably from 1/1 to 1/20, more preferably from 1/1 to 1/10, even more preferably from 1/1 to 1/5, from the viewpoints of increasing the extraction efficiency of lipid and reducing the production cost.

The lipid obtainable by the production method of the present invention are such that the content percentage of medium-chain fatty acids among all the constituent fatty acids in the lipid thus obtainable is preferably 15% by mass or more, more preferably 20% by mass or more, even more preferably 30% by mass or more, even more preferably 40% by mass or more, even more preferably 50% by mass or more, from the viewpoint of increasing the productivity of medium-chain fatty acids, and the content percentage may be, for example, from 15% to 100% by mass, from 30% to 85% by mass, or from 50% to 75% by mass. Furthermore, the content percentage of lauric acid among all the constituent fatty acids in the lipid thus obtainable is, from the viewpoint of increasing the productivity of lauric acid, preferably 6% by mass or more, more preferably 10% by mass or more, even more preferably 15% by mass or more, even more preferably 20% by mass or more, even more preferably 30% by mass or more, even more preferably 40% by mass or more, and the content percentage may be, for example, from 6% to 85% by mass, from 10% to 80% by mass, or from 15% to 75% by mass.

The lipid obtainable by the production method of the present invention are such that, from the viewpoint of increasing the productivity of medium-chain fatty acids, the content percentage of fatty acids having 16 to 22 carbon atoms among all the constituent fatty acids in the lipid is preferably 90% or less, more preferably 80% by mass or less, even more preferably 70% by mass or less, even more preferably 60% by mass or less, even more preferably 50% by mass or less, even more preferably 40% by mass or less, even more preferably 30% by mass or less, even more preferably 20% by mass or less, even more preferably 10% by mass or less.

Thus, when algae of the class Dinophyceae are cultured in a glycerin-containing medium, the content percentage of medium-chain fatty acids among all the constituent fatty acids in the lipid (productivity of medium-chain fatty acids), which is about 10% by mass in the absence of glycerin, can be increased to from 15% to 100%. That is, according to the present invention, there can be provided a method of increasing the productivity of medium-chain fatty acids to from 15% to 100% by mass, preferably from 30% to 85% by mass, more preferably from 50% to 75% by mass, by adding glycerin.

Furthermore, the content percentage of lauric acid among all the constituent fatty acids in the lipid (productivity of lauric acid), which is about 5% by mass in the absence of glycerin, can be increased to from 6% to 85% by mass. That is, according to the present invention, there can be provided a method of increasing the productivity of lauric acid to from 6% to 85% by mass, preferably from 10% to 80% by mass, more preferably from 15% to 75% by mass, by adding glycerin.

Therefore, regarding the production method of the present invention, it is preferable that the content percentage of medium-chain fatty acids among all the constituent fatty acids in the lipid be 15% by mass or more, or it is more preferable that the content percentage of lauric acid among all the constituent fatty acids in the lipid be 6% by mass or more.

Furthermore, the method of culturing algae of the class Dinophyceae in a glycerin-containing medium is useful as a method of increasing the productivity of medium-chain fatty acids along with an increase in the content percentage and/or the amount of production of medium-chain fatty acids in the algae, or as a method of increasing the productivity of lauric acid along with an increase in the content percentage and/or the amount of production of lauric acid.

Furthermore, the method of culturing algae of the class Dinophyceae in a glycerin-containing medium is useful as a method for producing algae having increased productivity of medium-chain fatty acids along with an increase in the content percentage and/or the amount of production of medium-chain fatty acids, or a method for producing algae having increased productivity of lauric acid along with an increase in the content percentage and/or the amount of production of lauric acid.

<Method for Producing Medium-Chain Fatty Acid Esters>

The ester production method of the present invention includes a step of subjecting the lipid obtained by the production method of the present invention as described above, to a transesterification reaction using an alcohol (hereinafter, may be referred to as “Step 3”). According to the ester production method of the present invention, medium-chain fatty acid esters can be produced efficiently.

The transesterification reaction can be carried out by a known method. The form of reaction may be any of a batch type and a continuous type; and, from the viewpoint of increasing the productivity and reducing the production cost, a continuous type is preferred. Furthermore, any of a tank type reactor having a stirrer and a fixed bed reactor packed with a catalyst may be used; and, from the viewpoint of reducing the burden of purification, it is preferable to use a fixed bed reactor that does not require catalytic separation.

Regarding the alcohol used in Step 3, it is preferable to use a lower alcohol having 1 to 5 carbon atoms from the viewpoint of increasing the productivity and reducing the production cost, and examples of the alcohol include methanol, ethanol, propanol and butanol. Industrially, methanol is preferred from the viewpoints of low cost and the ease of recovery.

The molar ratio of the alcohol with respect to the lipid (calculated by considering the entire lipid as triglycerides) for the transesterification reaction is preferably 1.5 times or more by mole, more preferably 2 times or more by mole, even more preferably 5 times or more by mole, of the stoichiometrically required amount, from the viewpoint of obtaining a satisfactory reaction rate. Furthermore, from the viewpoint of suppressing the amount of the raw material alcohol recovered and thereby performing the reaction in an economically efficient manner, the molar ratio of the alcohol is preferably 50 times or less by mole, more preferably 30 times or less by mole, even more preferably 15 times or less by mole. Also, from the viewpoint of obtaining a satisfactory reaction rate, and from the viewpoint of suppressing the amount of the raw material alcohol recovered and thereby performing the reaction in an economically efficient manner, the molar ratio of the raw material alcohol with respect to the lipid is preferably from 1.5 to 50 times by mole, more preferably from 2 to 30 times by mole, even more preferably from 5 to 15 times by mole.

Step 3 is preferably carried out in the presence of a catalyst, from the viewpoint of increasing the productivity. For the catalyst, a homogeneous alkali catalyst such as sodium hydroxide, potassium hydroxide or sodium alcoholate is generally used; and, solid catalysts such as an ion exchange resin, hydrous zirconium oxide, aluminum phosphate, sulfuric acid-supporting zirconium oxide and titanosilicate can also be used.

The amount of the catalyst used in the transesterification reaction is preferably 1% by mass or more, more preferably 3% by mass or more, even more preferably 5% by mass or more, with respect to the lipid from the viewpoint of increasing the reaction efficiency. Furthermore, from the viewpoint of maintaining a sufficiently suspended state by stirring, the amount of the catalyst used is preferably 20% by mass or less, more preferably 17% by mass or less, even more preferably 15% by mass or less, with respect to the lipid. Therefore, from the viewpoint described above, the amount of the catalyst used is preferably from 1% to 20% by mass, more preferably from 3% to 17% by mass, even more preferably from 5% to 15% by mass.

The reaction temperature for the transesterification reaction is preferably from 50° C. to 220° C., more preferably from 60° C. to 200° C., even more preferably from 80° C. to 200° C., even more preferably from 130° C. to 200° C., from the viewpoint of increasing the reaction efficiency and suppressing side products. Furthermore, the reaction pressure is preferably from 0.1 MPa to 10 MPa, more preferably from 0.5 MPa to 8 MPa, even more preferably from 2 MPa to 6 MPa, from the viewpoint of increasing the reaction efficiency.

<Method for Producing Medium-Chain Fatty Acids>

The method for producing medium-chain fatty acids of the present invention includes a step of hydrolyzing the lipid obtained by the production method of the present invention described above.

The method for hydrolyzing the lipid is not particularly limited, and any conventionally known method can be used (see, for example, “Shinpan Shibosan Kagaku (Fatty Acid Chemistry, New Edition)” (Saiwai Shobo Co., Ltd.)). Examples of industrially preferred hydrolysis methods include a high temperature high pressure decomposition method (see, for example, JP 2003-113395 A), an enzymatic decomposition method (see, for example, JP 2000-160188 A) and the like.

In addition, separation and purification of medium-chain fatty acids or medium-chain fatty acid esters from the mixed fatty acid esters or mixed fatty acids thus obtained can be carried out by conventional methods, for example, by using a column chromatography method and distillation.

In regard to the exemplary embodiment described above, the following embodiments of the present invention will be disclosed.

<1> A method for producing a lipid, the method comprising the following steps of (1) and (2):

(1) a step of culturing algae of the class Dinophyceae in a medium containing glycerin, to obtain a culture; and

(2) a step of collecting a lipid from the culture thus obtained.

<2> The method for producing a lipid according to <1>, wherein the lipid is a simple lipid, complex lipid or derivative lipid, preferably a simple lipid or complex lipid, more preferably a simple lipid, even more preferably an oil or fat.

<3> The method for producing a lipid according to <1> or

<2>, wherein the content percentage of medium-chain fatty acids among all the constituent fatty acids in the lipid is 15% by mass or more, preferably 20% by mass or more, more preferably 30% by mass or more, even more preferably 40% by mass or more, even more preferably 50% by mass or more.

<4> The method for producing a lipid according to any one of <1> to <3>, wherein the content percentage of lauric acid among all the constituent fatty acids in the lipid is 6% by mass or more, preferably 10% by mass or more, more preferably 15% by mass or more, even more preferably 20% by mass or more, even more preferably 30% by mass or more, even more preferably 40% by mass or more.

<5> The method for producing a lipid according to <1>, wherein the content percentage of fatty acids having 16 to 22 carbon atoms among all the constituent fatty acids in the lipid is 90% or less, preferably 80% by mass or less, more preferably 70% by mass or less, even more preferably 60% by mass or less, even more preferably 50% by mass or less, even more preferably 40% by mass or less, even more preferably 30% by mass or less, even more preferably 20% by mass or less, even more preferably 10% by mass or less.

<6> The method for producing a lipid according to any one of <1> to <5>, wherein the algae of the class Dinophyceae are algae of the order Noctilucales, the order Prorocentrales, the order Dinophysales, the order Gymnodiniales, the order Peridiniales, the order Gonyaulacales, the order Blastodiniales or the order Phytodiniales, preferably algae of the order Gymnodiniales.

<7> The method for producing a lipid according to <6>, wherein the algae of the order Gymnodiniales are algae belonging to the genus Amphidinium, Cochlodinium, Gymnodinium, Gyrodinium, Erythropsodinium, Hemidinium, Katodinium, Nematodinium, Oxyrrhis, Polykrikos, Torodinium, Symbiodinium, Warnowia, Woloszynskia or Zooxanthella, preferably algae belonging to the genus Symbiodinium.

<8> The method for producing a lipid according to <7>, wherein the algae belonging to the genus Symbiodinium are Symbiodinium microadriaticum, Symbiodinium goreaui, Symbiodinium linucheae, Symbiodinium bermudense, Symbiodinium meandrinae, Symbiodinium californium, Symbiodinium kawagutii, Symbiodinium corculorum, Symbiodinium consortia, Symbiodinium muscatinei, Symbiodinium freudenthal, Symbiodiniumpulchrorum, and Symbiodinium pilosum; preferably Symbiodinium microadriaticum; more preferably Symbiodinium microadriaticum strain LB2281 and Symbiodinium sp. strain NIES-2638.

<9> The method for producing a lipid according to any one of <1> to <8>, wherein the medium used for culturing the algae in Step 1 is Daigo IMK medium, f/2 medium, ESM medium, L1 medium or MNK medium; preferably f/2 medium, ESM medium or Daigo IMK medium; more preferably ESM medium or Daigo IMK medium; even more preferably Daigo INK medium.

<10> The method for producing a lipid according to any one of <1> to <9>, wherein the content of glycerin in the medium used in Step 1 is preferably 0.01% by mass or more, more preferably 0.02% by mass or more, even more preferably 0.05% by mass or more, even more preferably 0.07% by mass or more, even more preferably 0.08% by mass or more, even more preferably 0.09% by mass or more, even more preferably 0.1% by mass or more; is preferably 10% by mass or less, more preferably 5% by mass or less, even more preferably 4% by mass or less, even more preferably 3% by mass or less, even more preferably 2% by mass or less, even more preferably 1% by mass or less, even more preferably 0.5% by mass or less; and is from 0.01% to 10% by mass, preferably from 0.02% to 5% by mass, more preferably from 0.05% to 4% by mass, even more preferably from 0.07% to 3% by mass, even more preferably from 0.08% to 2% by mass, even more preferably from 0.09% to 2.0% by mass, even more preferably from 0.1% to 2.0% by mass, even more preferably from 0.1% to 1% by mass, even more preferably from 0.1% to 0.5% by mass.

<11> The method for producing a lipid according to any one of <1> to <10>, wherein the content of the nitrogen source in the medium used in Step 1 is, in terms of nitrogen atom equivalent, preferably 2 mg/L or more, more preferably 5 mg/L or more, even more preferably 10 mg/L or more, even more preferably 15 mg/L or more, even more preferably 20 mg/L or more, even more preferably 50 mg/L or more, even more preferably 100 mg/L or more; in terms of nitrogen atom equivalent, preferably 700 mg/L or less, more preferably 600 mg/L or less, even more preferably 500 mg/L or less, even more preferably 400 mg/L or less, even more preferably 300 mg/L or less, even more preferably 250 mg/L or less; and in terms of nitrogen atom equivalent, from 2 mg/L to 700 mg/L, preferably from 5 mg/L to 600 mg/L, more preferably from 10 mg/L to 500 mg/L, even more preferably from 15 mg/L to 400 mg/L, even more preferably from 20 mg/L to 300 mg/L, even more preferably from 50 mg/L to 250 mg/L, even more preferably from 100 mg/L to 250 mg/L.

<12> The method for producing a lipid according to any one of <1> to <11>, wherein the content of the phosphorus source in the medium used in Step 1 is, in terms of phosphorus atom equivalent, preferably 0.5 mg/L or more, more preferably 1 mg/L or more, even more preferably 2 mg/L or more, even more preferably 4 mg/L or more, even more preferably 8 mg/L or more; in terms of phosphorus atom equivalent, preferably 100 mg/L or less, more preferably 50 mg/L or less, even more preferably 25 mg/L or less, even more preferably 20 mg/L or less; and in terms of phosphorus atom equivalent, from 0.5 mg/L to 100 mg/L, more preferably from 1 mg/L to 50 mg/L, even more preferably from 2 mg/L to 25 mg/L, even more preferably from 4 mg/L to 20 mg/L, even more preferably from 8 mg/L to 20 mg/L.

<13> The method for producing a lipid according to any one of <1> to <12>, wherein the content of glucose in the medium used in Step 1 is 5% by mass or less, preferably 2% by mass or less, more preferably 1% by mass or less, even more preferably 0.5% by mass or less, even more preferably 0.1% by mass or less.

<14> The method for producing a lipid according to any one of <1> to <13>, wherein the pH of the medium used in Step 1 is in the range of from 5 to 10, preferably in the range of from 6 to 9, even more preferably in the range of from 7 to 8.

<15> The method for producing a lipid according to any one of <1> to <14>, wherein the amount of algae to be inoculated into the medium is from 1% to 10% (vol/vol), preferably from 1% to 5% (vol/vol), per medium.

<16> The method for producing a lipid according to any one of <1> to <15>, wherein the culture temperature for Step 1 is in the range of from 5° C. to 40° C., preferably from 10° C. to 30° C., more preferably from 15° C. to 25° C.

<17> The method for producing a lipid according to any one of <1> to <16>, wherein the culture of Step 1 is carried out under light irradiation.

<18> The method for producing a lipid according to <17>, wherein the illuminance at the time of light irradiation is in the range of from 100 Lux to 50,000 Lux, preferably in the range of from 300 Lux to 10,000 Lux, more preferably in the range of from 1,000 Lux to 6,000 Lux.

<19> The method for producing a lipid according to <17> or <18>, wherein the light irradiation is carried out with the light-dark cycle, and the cycle is from 8 hours to 24 hours, preferably from 10 hours to 18 hours, more preferably 12 hours.

<20> The method for producing a lipid according to any one of <1> to <17>, wherein the culture period after the addition of glycerin is from 3 days to 90 days, preferably from 3 days to 30 days, more preferably from 7 days to 30 days.

<21> A method for producing medium-chain fatty acid esters, the method comprising a step of subjecting the lipid obtained by the production method according to any one of <1> to <20> to a transesterification reaction with an alcohol.

<22> The method for producing medium-chain fatty acid esters according to <21>, wherein the alcohol used in the transesterification reaction is a lower alcohol having 1 to 5 carbon atoms, preferably methanol.

<23> The method for producing medium-chain fatty acid esters according to <21> or <22>, wherein a molar ratio of the alcohol with respect to the lipid (calculated by considering the entire lipid as triglycerides) for the transesterification reaction is from 1.5 times to 50 times by mole, preferably from 2 times to 30 times by mole, more preferably from 5 times to 15 times by mole.

<24> A method for producing medium-chain fatty acids, the method comprising hydrolyzing the lipid obtained by the production method according to any one of <1> to <20>.

<25> A method for increasing the productivity of the medium-chain fatty acids of algae of the class Dinophyceae, the method comprising culturing the algae in a medium containing glycerin.

<26> The method for increasing the productivity of the medium-chain fatty acids of algae according to <24>, wherein the content percentage of the medium-chain fatty acids among all the constituent fatty acids in the lipid (productivity of medium-chain fatty acids) is increased to from 15% to 100% by mass, preferably from 30% to 85% by mass, more preferably from 50% to 75% by mass.

<27> The method for increasing the productivity according to <25> or <26>, wherein the medium-chain fatty acid is lauric acid.

<28> A method for producing an alga having increased productivity of medium-chain fatty acids, the method comprising culturing algae of the class Dinophyceae in a medium containing glycerin.

<29> The method for producing an alga according to <28>, wherein the medium-chain fatty acid is lauric acid.

### EXAMPLES

In the following Examples and Comparative Examples, “%” means “mass %”. Various measurements were carried out by the following methods.

(1) Conditions for culturing algae

For the culture of algae, Daigo IMK medium (manufactured by NIHON PHARMACEUTICAL CO., LTD.) was used. The details of the medium composition will be presented in the following Table 1. A sterilized 100-mL conical flask (made of PYREX (registered trademark)) and a cotton plug (CS-28 manufactured by AS ONE Corporation.) were used as a culture vessel, and 50 mL of a medium that had been sterilized by filtering using a filter unit (manufactured by Nalgene Nunc) was dispensed therein. One milliliter of a culture fluid of algae that had been sub-cultured in advance using the same liquid medium was inoculated into fresh medium, and the algal cells were subjected to static culture under fluorescent lamp light at an illuminance of about 3,000 Lux at 20° C. under the conditions of a 12-hour light-dark cycle.

TABLE 1

| | for 1 L | |
|---|---|---|
| NaNO₃ | 200 | mg |
| Na₂HPO₄ | 1.4 | mg |
| K₂HPO₄ | 5 | mg |
| NH₄Cl | 2.68 | mg |
| Fe-EDTA | 5.2 | mg |
| Mn-EDTA | 332 | μg |
| Na2-EDTA | 37.2 | mg |
| ZnSO₄•7H₂O | 23 | μg |
| CoSO₄•7H₂O | 14 | μg |
| Na₂MoO₄•2H₂O | 7.3 | μg |
| CuSO₄•5H₂O | 2.5 | μg |
| H₂SeO₃ | 1.7 | μg |
| MnCl₂•4H₂O | 180 | μg |
| Thiamin•HCl | 200 | μg |
| Biotin | 1.5 | μg |
| Vitamin B₁₂ | 1.5 | μg |
| Artificial seawater | 35.96 | g |
| powder | | |

* In Table 1, the content of nitrogen sources in the medium was 33 mg/L in terms of nitrogen atom equivalents in the medium, and the content of phosphorus sources was 1.2 mg/L in terms of phosphorus atom equivalent.

(2) Method for Collecting Culture

Four milliliter of the culture obtained by culturing was centrifuged under the conditions of 3000 rpm for 30 minutes, and thus a sediment fraction was obtained. The sediment fraction thus obtained was dried at 80° C. for about 3 hours to 16 hours, and thus dried algal bodies were obtained.

(3) Method for Collecting Lipid

The weight of the dried algal bodies thus obtained was measured, and then the algal bodies were suspended in 0.5 mL of 1% brine. Fifty microliter of 7-pentadecanone at 1 mg/mL was added thereto as an internal standard, subsequently 0.5 mL of chloroform and 1 mL of methanol were added to the culture fluid, and the mixture was vigorously stirred and then left to stand for 30 minutes. Thereafter, 0.5 mL of chloroform and 0.5 mL of 1.5% KCl were added thereto, the mixture was stirred, and then the mixture was subjected to centrifugation at 3,000 rpm for 15 minutes. A chloroform layer (lower layer) was collected using a Pasteur pipette.

(4) Transesterification Reaction (Production of Fatty Acid Esters)

About 0.5 mL of the chloroform layer thus obtained was dried to solid by blowing nitrogen gas, 700 μL of a 0.5 N potassium hydroxide/methanol solution was added thereto, and the temperature of the mixture was kept constant at 80° C. for 30 minutes. Subsequently, 1 mL of a 14% boron trifluoride solution (manufactured by SIGMA Corporation.) was added thereto, and the temperature of the mixture was kept constant at 80° C. for 20 minutes. Subsequently, 1 mL each of hexane and saturated brine were added thereto, the mixture was left to stand for 30 minutes at room temperature, and then a hexane layer as an upper layer was collected and dried. Thus, fatty acid esters were obtained.

(5) Method for Identification of Fatty Acid Esters and Measurement of Total Amount of Fatty Acids, and Fatty Acid Content

Identification of fatty acid esters, the total amount of fatty acids and the fatty acid content were obtained by a gas chromatographic (GC) analysis under the conditions described below.

Identification of fatty acid esters was determined based on whether their retention times were identical with the retention times of the standard substances described below. Also, the amounts of fatty acid esters detected by the GC analysis were calculated based on the internal standards, and the sum of amount was designated as the total amount of fatty acids. Furthermore, a value obtained by dividing the total amount of fatty acids by the amount of dried algal bodies, and multiplying the resultant by 100 was designated as the fatty acid content (%)

Apparatus: 6890 A (manufactured by Agilent, Inc.) Column: DB-1 MS 30 m×200 μm×0.25 μm (manufactured by J&W Scientific, Inc.)

Mobile phase; high purity helium (flow rate 1 mL/min) Temperature rise program: 150° C. (0.5 minutes), 40° C./min, 320° C. (4 minutes)

Injection port detector temperature: 300° C.

Injection method: split mode (split ratio=75:1)

Amount of sample injected: 5 μl

Column flow rate: 0.3 mL/min (constant)

Detector: FID

Carrier gas: hydrogen

Makeup gas: helium

Standard substance: Fatty acid esters manufactured by SIGMA Corporation, as described below, were used.

Methyl laurate (C12), methyl myristate (C14), methyl palmitate (C16) and methyl stearate (C18); and as unsaturated fatty acids, methyl palmitoleate (C16:1), methyl oleate (C18:1), methyl linoleate (C18:2), methyl linolenate (C18:3), methyl eicosapentaenoate (C20:5) and methyl docosahexaenoate (C22:6).

(6) Measurement of Number of Cells

Measurement of the number of cells was carried out using a counting chamber (AS ONE Corporation; Thoma's erythrocytometer standard) by adding 1/50 volume of Lugol solution (50 mg/mL iodine and 100 mg/mL potassium iodide) to an appropriately diluted sample, immobilizing the cells, and subsequently measuring the number of cells using a stereoscopic microscope (CKX31, manufactured by Olympus Corporation) or a biological microscope (ECLIPSE 80i, manufactured by Nikon Corporation).

### Example 1

As algae of the class Dinophyceae, Zooxanthella microadriatica strain LB2281 (Symbiodinium microadriaticum strain LB2281) obtained from UTEX (The Culture Collection of Algae at University of Texas at Austin) was used.

Glycerin was added to a medium that had been cultured for 3 weeks after the initiation of culture, so that a final concentration was 0.5%, and the cells were further cultured for one week, and then the amounts of fatty acids and the kinds of fatty acids were determined by a GC analysis. The analysis results are presented in Table 2.

### Comparative Examples 1 and 2

Cells were cultured under the same conditions as in Example 1, except that glycerin was not added, or glucose was added instead of glycerin, so that a final concentration was 0.5%. The analysis results are presented in Table 2.

TABLE 2

| | | | | | | | | | | | | Percentage |
|---|---|---|---|---|---|---|---|---|---|---|---|---|
| | | Fatty acid | | | | | | | | | | of medium-chain |
| | | productivity | Fatty acid composition (%) | | | | | | | | | fatty acids (%) |
| | | (mg/L) | C10:0 | C12:0 | C14:0 | C16:0 | C16:1 | C18:n | C20:n | C22:n | others | C10-C14 |
| Example 1 | 0.5% Glycerin | 90.7 | 0.9 | 10.0 | 12.6 | 19.2 | 2.6 | 39.2 | 5.0 | 10.7 | 1.0 | 23.5 |
| | added | | | | | | | | | | | |
| Comparative | Control | 24.8 | 0.0 | 2.0 | 4.1 | 19.4 | 2.4 | 51.8 | 7.8 | 11.5 | 0.9 | 6.1 |
| Example 1 | (no addition) | | | | | | | | | | | |
| Comparative | 0.5% Glucose | 61.8 | 0.0 | 1.5 | 3.7 | 16.1 | 2.2 | 55.3 | 8.4 | 11.7 | 1.2 | 5.2 |
| Example 2 | added | | | | | | | | | | | |

As shown in Table 2, in Example 1 in which glycerin was added to the medium, a marked increase in the amounts of fatty acids and an increase in the ratio of medium-chain fatty acids among all the fatty acids were recognized.

### Examples 2 to 5

Cells were cultured under the same conditions as in Example 1, except that glycerin was added so that a final concentration was 0.1%, 0.5%, 1.0% or 2.0%, and the cells were culture for 2 weeks. The analysis results are presented in Table 3.

### Comparative Examples 3 and 4

Cells were cultured under the same conditions as in Example 2, except that glycerin was not added, or glucose was added instead of glycerin so that a final concentration was 0.5%. The analysis results are presented in Table 3.

TABLE 3

| | | | | | | | | | | | | Percentage |
|---|---|---|---|---|---|---|---|---|---|---|---|---|
| | | Fatty acid | | | | | | | | | | of medium-chain |
| | | productivity | Fatty acid composition (%) | | | | | | | | | fatty acids (%) |
| | | (mg/L) | C10:0 | C12:0 | C14:0 | C16:0 | C16:1 | C18:n | C20:n | C22:n | others | C10-C14 |
| Example 2 | 0.1% Glycerin | 64.5 | 0.7 | 10.2 | 13.9 | 21.4 | 3.2 | 31.4 | 4.7 | 9.9 | 5.7 | 24.8 |
| | added | | | | | | | | | | | |
| Example 3 | 0.5% Glycerin | 50.8 | 1.0 | 10.8 | 13.6 | 21.6 | 3.4 | 34.2 | 3.9 | 10.1 | 1.5 | 25.4 |
| | added | | | | | | | | | | | |
| Example 4 | 1.0% Glycerin | 33.0 | 0.0 | 8.1 | 11.8 | 23.3 | 3.8 | 35.5 | 5.0 | 9.9 | 2.5 | 19.9 |
| | added | | | | | | | | | | | |
| Example 5 | 2.0% Glycerin | 29.3 | 0.9 | 6.1 | 8.8 | 22.6 | 2.5 | 43.3 | 3.7 | 7.4 | 4.7 | 15.8 |
| | added | | | | | | | | | | | |
| Comparative | Control | 43.2 | 0.0 | 5.3 | 8.4 | 20.6 | 2.5 | 42.5 | 7.3 | 10.2 | 3.3 | 13.7 |
| Example 3 | (no addition) | | | | | | | | | | | |
| Comparative | 0.5% Glucose | 59.9 | 0.0 | 3.2 | 5.2 | 18.2 | 2.8 | 48.1 | 8.5 | 12.7 | 1.3 | 8.4 |
| Example 4 | added | | | | | | | | | | | |

As shown in Table 3, an increase in the ratio of medium-chain fatty acids was recognized at a glycerin concentration in the medium in the range of 0.1% to 2.0% by mass.

### Example 6

Cells were cultured under the same conditions as in Example 1, except that cells of strain LB2281 that had been sterilized by a micropipetting method were used and the cells were cultured for 4 weeks by adding glycerin so that a final concentration was 1.0% from the initiation stage of culture. The analysis results are presented in Table 4.

### Comparative Examples 5 to 11

Cells were cultured under the same conditions as in Example 6, except that glycerin was not added (Comparative Example 5), or glucose (Comparative Example 6), fructose (Comparative Example 7), sucrose (Comparative Example 8), xylose (Comparative Example 9), sorbitol (Comparative Example 10) or mannitol (Comparative Example 11) was added instead of glycerin. The analysis results are presented in Table 4.

TABLE 4

| | | | | | | | | | | | Percentage of |
|---|---|---|---|---|---|---|---|---|---|---|---|
| | | | Fatty acid | | | | | | | | medium-chain |
| | | Number of cells | productivity | Fatty acid composition (%) | | | | | | | fatty acids (%) |
| | | (*10⁶ cells/mL) | (mg/L) | C12:0 | C14:0 | C16:0 | C16:1 | C18:n | C20:n | C22:n | C12-C14 |
| Example 6 | 1.0% | 1.05 | 66.4 | 7.4 | 12.6 | 25.0 | 4.5 | 30.3 | 3.1 | 17.1 | 20.0 |
| | Glycerin added | | | | | | | | | | |
| Comparative | Control (no | 0.94 | 38.2 | 2.3 | 5.1 | 24.1 | 3.3 | 43.0 | 6.5 | 15.7 | 7.4 |
| Example 5 | addition) | | | | | | | | | | |
| Comparative | 1.0% Glucose | 0.92 | 36.1 | 2.6 | 5.2 | 22.6 | 3.2 | 43.7 | 7.0 | 15.7 | 7.8 |
| Example 6 | added | | | | | | | | | | |
| Comparative | 1.0% | 0.88 | 13.4 | 2.2 | 4.1 | 22.3 | 3.0 | 50.0 | 4.0 | 14.3 | 6.4 |
| Example 7 | Fructose added | | | | | | | | | | |
| Comparative | 1.0% Sucrose | 0.90 | 30.8 | 3.0 | 6.1 | 23.7 | 3.4 | 40.9 | 6.7 | 16.2 | 9.1 |
| Example 8 | added | | | | | | | | | | |
| Comparative | 1.0% Xylose | 0.96 | 25.7 | 2.3 | 4.3 | 21.8 | 3.1 | 46.2 | 5.5 | 16.8 | 6.6 |
| Example 9 | added | | | | | | | | | | |
| Comparative | 1.0% | 0.87 | 30.7 | 2.3 | 4.8 | 24.0 | 3.4 | 43.9 | 7.3 | 14.2 | 7.1 |
| Example 10 | Sorbitol added | | | | | | | | | | |
| Comparative | 1.0% | 0.88 | 31.0 | 2.4 | 4.6 | 22.8 | 3.2 | 45.0 | 7.6 | 14.4 | 6.9 |
| Example 11 | Mannitol added | | | | | | | | | | |

As shown in Table 4, when compounds other than glycerin were added, the effect of increasing the number of cells was not recognized. Furthermore, when glycerin was added, a noticeable increase in the amounts of fatty acids and an increase in the ratio of medium-chain fatty acids were recognized.

### Example 7

Cells were cultured under the same conditions as in Example 4, except that the culture period after the addition of glycerin was adjusted to 4 weeks, 5 weeks, 7 weeks, 8 weeks or 12 weeks. The culture fluid (4 mL) was collected on the 4^{th} week, 5^{th} week, 7^{th} week, 8^{th} week or 12^{th} week of culture, and a GC analysis of each of the culture fluid was conducted. The analysis results are presented in Table 5.

TABLE 5

| | | | | | | | | | | | Percentage of |
|---|---|---|---|---|---|---|---|---|---|---|---|
| 1.0% | Fatty acid | | | | | | | | | | medium-chain |
| Glycerin | productivity | Fatty acid composition (%) | | | | | | | | | fatty acids (%) |
| added | (mg/L) | C10:0 | C12:0 | C14:0 | C16:0 | C16:1 | C18:n | C20:n | C22:n | others | C10-C14 |
| 4^{th} week | 71.1 | 0.5 | 4.6 | 7.0 | 23.8 | 5.6 | 34.2 | 2.9 | 19.5 | 1.9 | 12.1 |
| 5^{th} week | 93.0 | 0.8 | 8.2 | 11.4 | 24.1 | 5.0 | 36.4 | 1.9 | 11.6 | 0.6 | 20.4 |
| 7^{th} week | 195.0 | 0.6 | 9.5 | 10.8 | 25.4 | 5.6 | 32.3 | 1.3 | 13.8 | 0.7 | 20.9 |
| 8^{th} week | 178.5 | 0.9 | 11.3 | 12.1 | 24.9 | 6.1 | 27.2 | 1.3 | 15.4 | 0.7 | 24.3 |
| 12^{th} week | 362.8 | 0.0 | 8.0 | 11.4 | 22.0 | 7.7 | 19.8 | 1.4 | 28.5 | 1.3 | 19.4 |

As shown in Table 5, in all of the glycerin-added systems on the 4^{th} week to 12^{th} week of culturing, a noticeable increase in the amounts of fatty acids and an increase in the ratio of medium-chain fatty acids in all the fatty acids were recognized.

### Example 8

Cells were cultured under the same conditions as in Example 6, except that the cells were cultured for 6 weeks. The analysis results are presented in Table 6.

### Example 9

Cells were cultured under the same conditions as in Example 6, except that in IMK medium (Table 1), the concentration of sodium nitrate (NaNO₃) was changed to 1000 mg/L, the concentration of disodium phosphate (Na₂HPO₄) was changed to 14 mg/L and the concentration of dipotassium phosphate (K₂HPO₄) was changed to 50 mg/L (the content of nitrogen sources in the medium was 165 mg/L in terms of nitrogen atom equivalent, and the content of phosphorus sources was 12 mg/L in terms of phosphorus atom equivalent) and the cells were cultured for 6 weeks. The analysis results are presented in Table 6.

### Comparative Example 12

Cells were cultured under the same conditions as in Example 8, except that glycerin was not added. The analysis results are presented in Table 6.

### Comparative Example 13

Cells were cultured under the same conditions as in Example 9, except that glycerin was not added. The analysis results are presented in Table 6.

TABLE 6

| | | | | | | | | | | Percentage of |
|---|---|---|---|---|---|---|---|---|---|---|
| | | Fatty acid | | | | | | | | medium-chain |
| | | productivity | Fatty acid composition (%) | | | | | | | fatty acids (%) |
| | | (mg/L) | C12:0 | C14:0 | C16:0 | C16:1 | C18:n | C20:n | C22:n | C12-C14 |
| Example 8 | IMK medium | 338.7 | 18.8 | 20.7 | 22.3 | 5.3 | 14.7 | 0.6 | 17.6 | 39.5 |
| | 1.0% glycerin added | | | | | | | | | |
| Example 9 | N,P-enriched IMK | 586.4 | 25.7 | 22.3 | 17.4 | 6.7 | 10.0 | 1.4 | 16.6 | 48.0 |
| | medium | | | | | | | | | |
| | 1.0% glycerin added | | | | | | | | | |
| Comparative | IMK medium | 75.5 | 5.2 | 10.9 | 31.8 | 7.6 | 32.2 | 0 | 12.4 | 16.1 |
| Example 12 | No glycerin added | | | | | | | | | |
| Comparative | N,P-enriched IMK | 13.6 | 1.3 | 2.6 | 45.2 | 0 | 49.6 | 0 | 1.3 | 3.9 |
| Example 13 | medium | | | | | | | | | |
| | No glycerin added | | | | | | | | | |

As shown in Table 6, when glycerin was incorporated in a medium enriched with nitrogen and phosphorus, a noticeable increase in the amounts of fatty acids and a noticeable increase in the ratio of medium-chain fatty acids in all the fatty acids were recognized.

### Example 10

As algae of the class Dinophyceae, culturing was initiated in the same manner as in Example 1 using Symbiodinium sp. strain NIES-2638 obtained from NIES (National Institute for Environmental Studies), and after the passage of 4 weeks, glycerin was added to the medium so that a final concentration was 0.5%. The cells were cultured for another 17 days, and then the amounts of fatty acids and the kinds of fatty acids were measured by a GC analysis. The analysis results are presented in Table 7.

### Comparative Example 14

Cells were cultured under the same conditions as in Example 10, except that glycerin was not added thereto. The analysis results are presented in Table 7.

TABLE 7

| | | | | | | | | | | Percentage of |
|---|---|---|---|---|---|---|---|---|---|---|
| | | | | | | | | | | medium-chain |
| | | Fatty acid | | | | | | | | fatty acids |
| | | productivity | Fatty acid composition (%) | | | | | | | (%) |
| | | (mg/L) | C12:0 | C14:0 | C16:0 | C16:1 | C18:n | C20:n | C22:n | C12-C14 |
| Example 10 | IMK medium, 0.5% | 146.6 | 4.1 | 13.5 | 6.8 | 29.0 | 24.7 | 2.0 | 19.8 | 17.6 |
| | glycerin added | | | | | | | | | |
| Comparative | IMK medium, no | 58.3 | 1.6 | 7.1 | 5.5 | 31.5 | 32.9 | 4.9 | 16.5 | 8.7 |
| Example 14 | glycerin added | | | | | | | | | |

As shown in Table 7, also for Symbiodinium sp. strain NIES-2638, a noticeable increase in the amounts of fatty acids and a noticeable increase in the ratio of medium-chain fatty acids in all the fatty acids were recognized as a result of the addition of glycerin.

### Example 11

Cells were cultured under the same conditions as in Example 10, except that Heterocapsa niei strain NIES-420 that belongs to the order Peridiniales obtained from NIES (National Institute for Environmental Studies) was used as algae of the class Dinophyceae and the cells were cultured for 7 days after the addition of glycerin. The analysis results are presented in Table 8.

### Comparative Example 15

Cells were cultured under the same conditions as in Example 11, except that glycerin was not added. The analysis results are presented in Table 8.

TABLE 8

| | | | | | | | | | | Percentage of |
|---|---|---|---|---|---|---|---|---|---|---|
| | | | | | | | | | | medium-chain |
| | | Fatty acid | | | | | | | | fatty acids |
| | | productivity | Fatty acid composition (%) | | | | | | | (%) |
| | | (mg/L) | C12:0 | C14:0 | C16:0 | C16:1 | C18:n | C20:n | C22:n | C12-C14 |
| Example 11 | IMK medium, 0.5% | 31.2 | 2.0 | 20.2 | 2.0 | 25.0 | 42.7 | 1.1 | 7.1 | 22.2 |
| | glycerin added | | | | | | | | | |
| Comparative | IMK medium, no | 21.8 | 0 | 7.5 | 1.3 | 20.6 | 50.3 | 4.8 | 15.6 | 7.5 |
| Example 15 | glycerin added | | | | | | | | | |

As shown in Table 8, also for the dinoflagellate Heterocapsa niei strain NIES-420, an increase in the amounts of fatty acids and a noticeable increase in the ratio of medium-chain fatty acids in all the fatty acids were recognized as a result of the addition of glycerin.

What is claimed is:

1. A method for producing a lipid, the method comprising the following steps (1) and (2):
(1) culturing algae of the class Dinophyceae in a medium containing glycerin to obtain a culture; and
(2) collecting a lipid from the culture thus obtained.

2. The method for producing a lipid according to claim 1, wherein the lipid is an oil or fat.

3. The method for producing a lipid according to claim 1, wherein the content percentage of medium-chain fatty acids in all the constituent fatty acids in the lipid is 15% by mass or more.

4. The method for producing a lipid according to claim 1, wherein the content percentage of lauric acid among all the constituent fatty acids in the lipid is 6% by mass or more.

5. The method for producing a lipid according claim 1, wherein the algae of the class Dinophyceae are algae of the order Gymnodiniales.

6. The method for producing a lipid according to claim 5, wherein the algae of the order Gymnodiniales are algae belonging to the genus Symbiodinium.

7. The method for producing a lipid according to claim 6, wherein the algae belonging to the genus Symbiodinium are Symbiodinium microadriaticum.

8. The method for producing a lipid according to claim 1, wherein the content of glycerin in the medium used in step (1) is from 0.01% to 10% by mass.

9. The method for producing a lipid according to claim 1, wherein the medium used in step (1) comprises a nitrogen source and the content of the nitrogen source in the medium is from 10 mg/L to 500 mg/L in terms of nitrogen atom equivalent.

10. The method for producing a lipid according to claim 1, wherein the medium used in step (1) comprises a phosphorus source and the content of the phosphorus source in the medium is from 0.5 mg/L to 100 mg/L in terms of phosphorus atom equivalent.

11. The method for producing a lipid according to claim 1, wherein the culture of step (1) is carried out under light irradiation.

12. A method for producing medium-chain fatty acid esters, the method comprising subjecting, to a transesterification reaction using an alcohol, a lipid obtained by a method comprising the following steps (1) and (2):
(1) culturing algae of the class Dinophyceae in a medium containing glycerin to obtain a culture; and
(2) collecting a lipid from the culture thus obtained.

13. A method for producing medium-chain fatty acids, the method comprising hydrolyzing a lipid obtained by a method comprising the following steps (1) and (2):
(1) culturing algae of the class Dinophyceae in a medium containing glycerin to obtain a culture; and
(2) collecting a lipid from the culture thus obtained.

14-17. (canceled)

18. The method for producing a lipid according to claim 1, wherein the content percentage of fatty acids having 16 to 22 carbon atoms among all the constituent fatty acids in the lipid is 90% or less.

19. The method for producing a lipid according to claim 1, wherein the medium used in step (1) comprises glucose and the content of glucose in the medium is 5 mass % or less.

20. The method for producing a lipid according to claim 1, wherein the amount of algae to be inoculated into the medium is from 1 to 10% (vol/vol).

21. The method for producing a lipid according to claim 1, wherein the culture period after the addition of glycerin is from 3 to 90 days.

22. The method of producing medium-chain fatty acid esters of claim 12, wherein the alcohol used in the transesterification is methanol.

23. The method of producing medium-chain fatty acid esters of claim 12, wherein a molar ratio of the alcohol with respect to the lipid in the transesterification is from 1.5 to 50 times by mole, and wherein the molar ratio is calculated by considering the entire lipid as triglyceride.

24. The method for producing medium-chain fatty acid esters of claim 12, wherein the medium used in step (1) comprises glucose and the content of glucose in the medium is 5 mass % or less.
